(19) United States
(12) Patent Application Publication
(10) Pub. No.: US 2022/0410409 A1
(43) Pub. Date: Dec. 29, 2022

(54) MECHANICAL COLLAR AND ASSOCIATED SYSTEMS AND METHODS

(71) Applicant: James L. Orrington, II D.D.S., P.C., Chicago, IL (US)

(72) Inventors: James L. Orrington, II, Flossmoor, IL (US); Michael Prince, Chicago, IL (US); Hyunchul Kim, Chicago, IL (US)

(21) Appl. No.: 17/746,573

(22) Filed: May 17, 2022

Related U.S. Application Data
(63) Continuation of application No. 29/832,468, filed on Mar. 28, 2022.
(60) Provisional application No. 63/228,251, filed on Aug. 2, 2021.
(60) Provisional application No. 63/215,188, filed on Jun. 25, 2021.

(51) Int. Cl. B25J 17/00

(52) U.S. Cl. CPC B25J 17/00

(57) ABSTRACT

Disclosed herein are mechanical collars comprising a first connecting component and a second connecting component adjoined to one another. The second connecting component may comprise two substantially circular passages, wherein the inner diameter of the first substantially circular passage is larger than the outer diameter of the second substantially circular passage. Also disclosed herein are systems for mitigating the risk of transmission of infectious disease, such as in a healthcare provider's office, comprising mechanical collars as described herein, protective apparatuses, and a mechanical arm such as, without limitation, an articulating mechanical arm. Also disclosed herein are associated methods of mitigating risk of transmission of infectious disease utilizing the mechanical collars and related systems described herein.

### CROSS-REFERENCES TO RELATED APPLICATIONS

The present application claims the benefit of and priority to U.S. Provisional Patent Application Nos. 63/228,251 and 63/215,188. The present application claims the benefit of and priority to U.S. Design patent application No. 29/832,468. The contents of each of the above-referenced patent applications are incorporated herein by reference in their entirety.

### FIELD OF THE INVENTION

The present disclosure is directed to mechanical collars that may be utilized to connect two objects. Mechanical collars as discussed herein may be utilized, without limitation, in a healthcare setting. Mechanical collars as discussed herein may be utilized, without limitation, in a dentist's office. Mechanical collars as discussed herein may be utilized to connect an articulating mechanical arm and a protective apparatus, which protective apparatus may comprise a substantially transparent shield component. When a mechanical collar of the present disclosure is utilized in this manner, the articulating mechanical arm may be utilized to position the protective apparatus, such as to position the protective apparatus so that the substantially transparent shield component thereof may be located over the head and neck region of a patient in a healthcare provider's office (e.g., a patient seated in a chair in a dentist's office). Such protective apparatuses, when utilized in this manner, may mitigate the risk of transmission of infectious disease to a patient from, or from a patient to, others in close proximity to the patient.

### SUMMARY

Mechanical collars enabled by this disclosure may comprise a first connecting component and a second connecting component adjoined to one another. The first connecting component may have an inner diameter of between approximately one inch and approximately five inches. The second connecting component may comprise a first substantially circular passage and a second substantially circular passage. The second substantially circular passage may be located within the first substantially circular passage. According to such an embodiment, the second substantially circular passage may have an outer diameter less than the inner diameter of the first substantially circular passage. According to such an embodiment, the first substantially circular passage may have an inner diameter of between approximately one inch and approximately six inches, without limitation. Mechanical collars enabled by the present disclosure may further comprise a C-clamp. Mechanical collars enabled by the present disclosure may further comprise a covering that may be positioned adjacent to the second connecting component. Mechanical collars enabled by the present disclosure may be constructed of steel, metal, polycarbonate, polyphenylsulfone, or polyethylene, without limitation.

Mechanical collars enabled by the present disclosure may be utilized in connection with systems useful for mitigating risk of transmission of an infectious disease. Such systems may comprise, in addition to mechanical collars of the present disclosure, a protective apparatus and a mechanical arm, such as an articulating mechanical arm. The protective apparatus may be operatively attached to the first connecting component of the mechanical collar component. An articulating mechanical arm component may be operatively attached to the second connecting component of the mechanical collar component. The present disclosure enables associated methods of using these systems to mitigate risk of transmission of infectious disease.

### BRIEF DESCRIPTION OF THE DRAWINGS

FIG. 1 is a left side view of a mechanical collar of the present disclosure connected to a protective apparatus.

FIG. 2 is a rear view of a mechanical collar of the present disclosure connected to a protective apparatus.

FIG. 3 is a right side view of a mechanical collar of the present disclosure connected to a protective apparatus.

FIG. 4 is a top plan view of a mechanical collar of the present disclosure connected to a protective apparatus.

FIG. 5 is a front view of a mechanical collar of the present disclosure connected to a protective apparatus.

FIG. 6 is a bottom plan view of a mechanical collar of the present disclosure connected to a protective apparatus.

FIG. 7 is a front top perspective view of a mechanical collar of the present disclosure connected to a protective apparatus.

FIG. 8 is a rear top perspective view of a mechanical collar of the present disclosure connected to a protective apparatus.

FIG. 9 is a front top perspective view of a mechanical collar of the present disclosure connected to a protective apparatus.

FIG. 10 is a bottom plan view of a mechanical collar of the present disclosure connected to a protective apparatus.

FIG. 11 is a rear perspective view of a mechanical collar of the present disclosure.

FIG. 12 is a rear top perspective view of a mechanical collar of the present disclosure.

FIG. 13 is a rear perspective view of a mechanical collar of the present disclosure.

FIG. 14 is a rear perspective view of a mechanical collar of the present disclosure.

FIG. 15 is a rear view of a mechanical collar of the present disclosure.

FIG. 16 is a top plan view of a mechanical collar of the present disclosure.

FIG. 17 is an exploded view of a mechanical collar of the present disclosure connected to a protective apparatus.

FIG. 18 is an exploded of a mechanical collar of the present disclosure.

### DETAILED DESCRIPTION

The following disclosure is provided to describe various embodiments of mechanical collars and associated systems and methods utilizing such mechanical collars. Skilled artisans will appreciate additional embodiments and uses of the mechanical collars and the associated systems and methods that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings.

For the purpose of clearly describing the components and features discussed throughout this disclosure, some frequently used terms will now be defined, without limitation. The term “protective apparatus” at it is used throughout this disclosure shall mean an apparatus comprising a protective barrier, such as a shield (e.g., a substantially transparent shield). The term “approximately” as it is used throughout this disclosure shall mean “about” and “close to” and should be interpreted as encompassing a range of values 10% above and 10% below the value with respect to which “approximately” is used.

Various aspects of the disclosure will now be described in detail, without limitation. In the following disclosure, mechanical collars, and associated systems and methods utilizing such mechanical collars, will be discussed. Those of skill in the art will appreciate that alternative labeling of the mechanical collars and the associated systems and methods described herein may be provided, which is consistent with the scope and spirit of this disclosure. Skilled readers should not view the inclusion of any alternative labels as limiting in any way.

The present disclosure is directed to mechanical collars that may be utilized to connect two objects. Mechanical collars as discussed herein may be utilized, without limitation, in a healthcare setting. Mechanical collars as discussed herein may be utilized, without limitation, in a dentist's office. Mechanical collars as discussed herein may be utilized to connect an articulating mechanical arm and a protective apparatus, which protective apparatus may comprise a substantially transparent shield component. When a mechanical collar of the present disclosure is utilized in this manner, the articulating mechanical arm may be utilized to position the protective apparatus, such as to position the protective apparatus so that the substantially transparent shield component thereof may be located over the head and neck region of a patient in a healthcare provider's office (e.g., a patient seated in a chair in a dentist's office). Such protective apparatuses, when utilized in this manner, may mitigate the risk of transmission of infectious disease to a patient from, or from a patient to, others in close proximity to the patient.

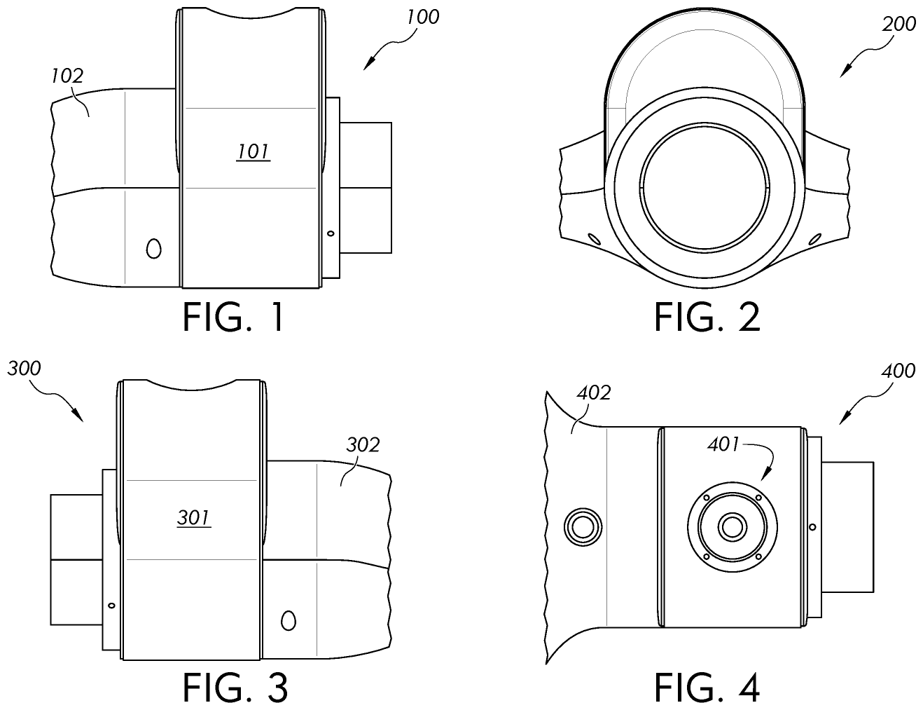

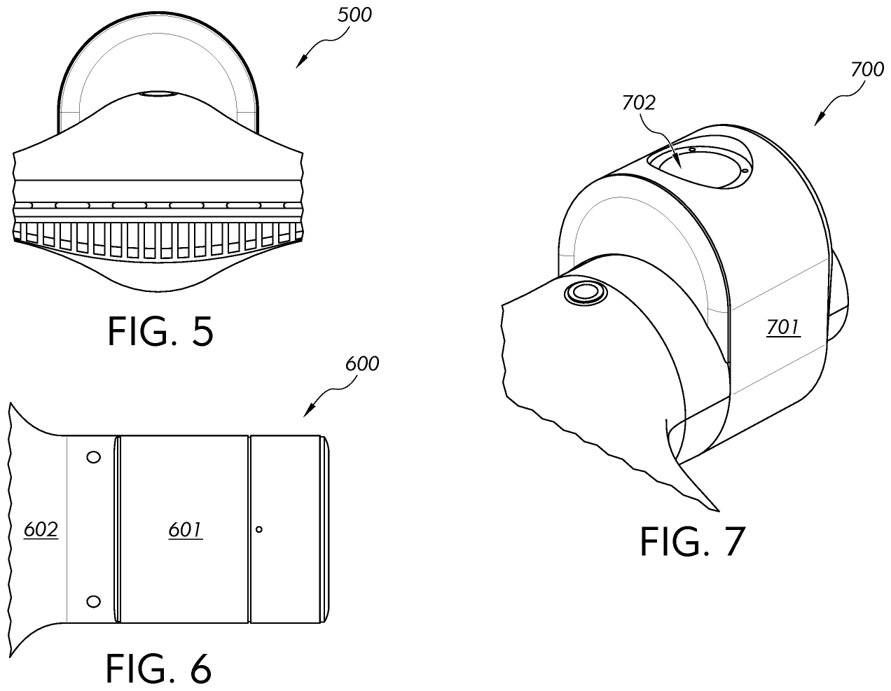

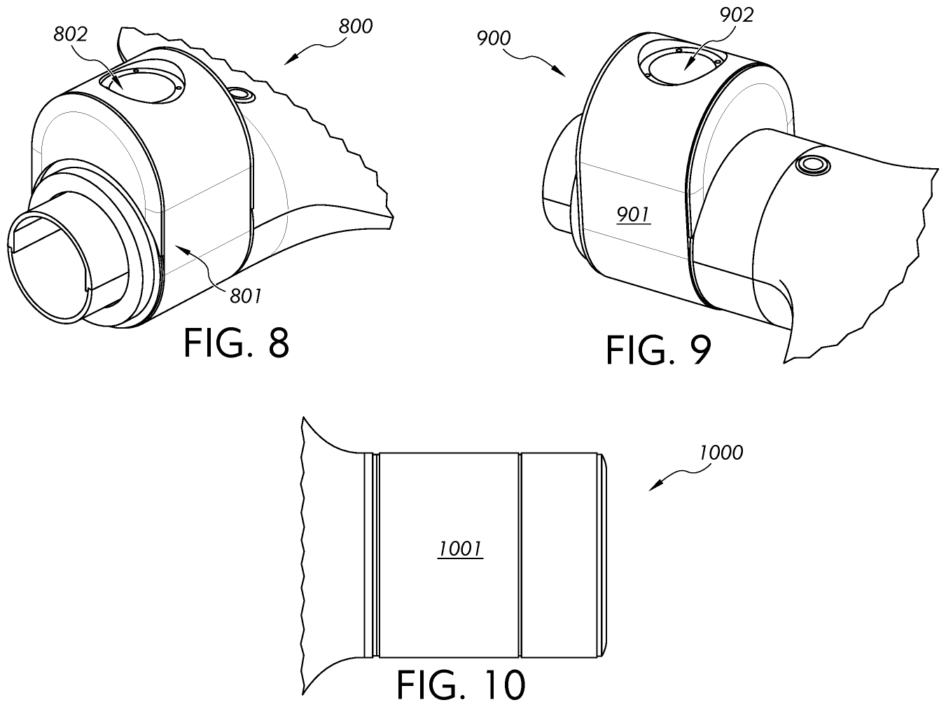

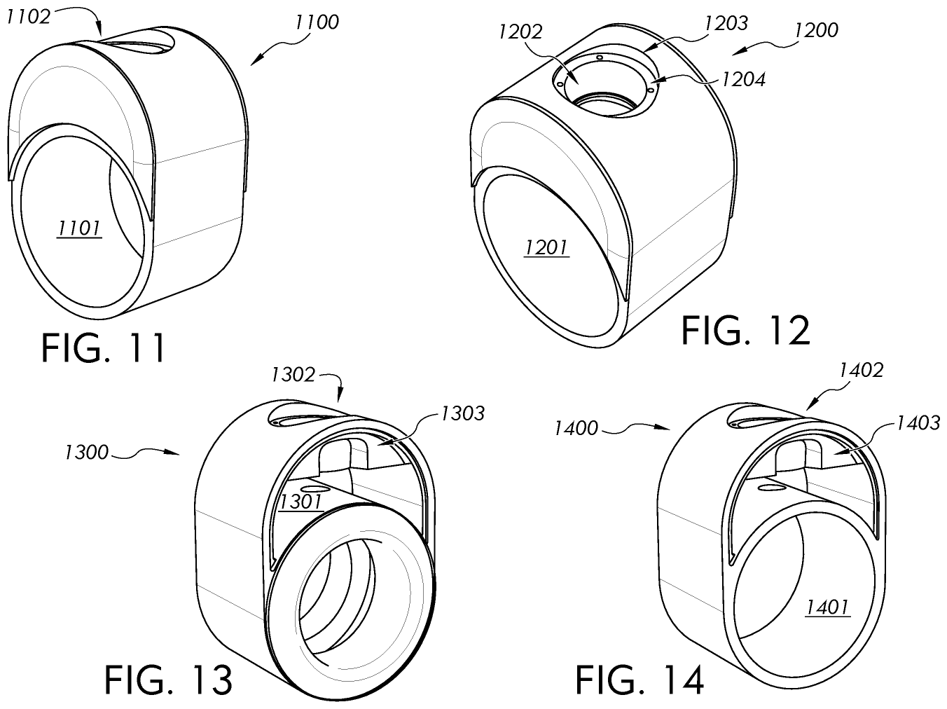

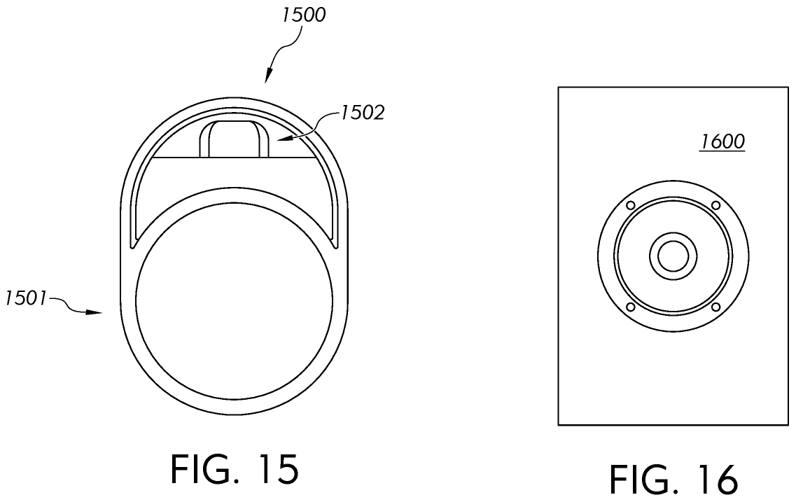

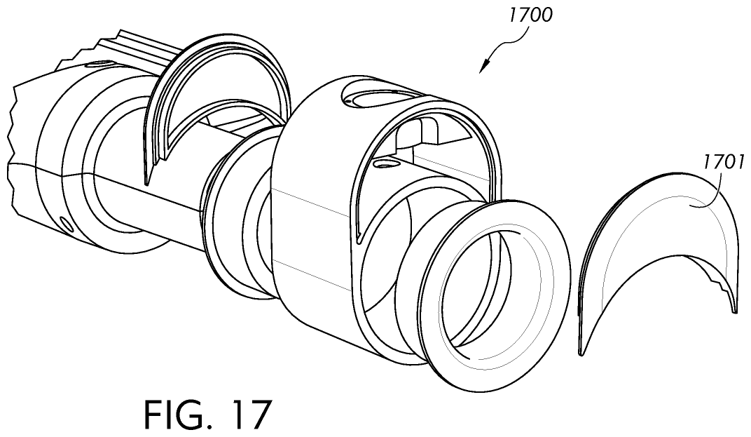

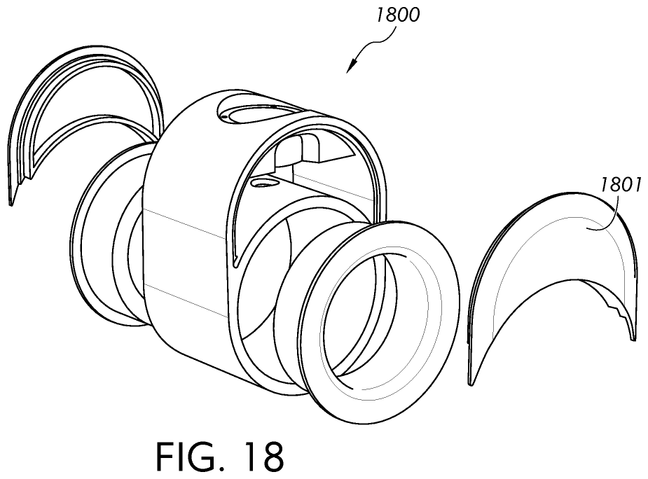

FIGS. 1-18 of the present disclosure comprise a series of graphic renderings of a mechanical collar 100, 200, 300, 400, 500, 600, 700, 800, 900, 1000, 1100, 1200, 1300, 1400, 1500, 1700, 1800 enabled by the present disclosure.

Said mechanical collars may comprise a first connecting component and a second connecting component.

Mechanical collars enabled by the present disclosure may be made out of any number of suitable materials, as would be readily appreciated by those skilled in the art. Without limitation, mechanical collars enabled by the present disclosure may be made of steel, metal (for example, aluminum), or other material of high tensile strength such as polycarbonate, polyphenylsulfone, ultra high molecular weight polyethylene, and other strong plastics.

FIGS. 1-10 and 17 submitted herewith show use of a mechanical collar according to the present disclosure in connection with a protective apparatus.

As noted, mechanical collars enabled by the present disclosure may comprise a first connecting component 101, 301, 601, 701, 801, 901, 1001, 1101, 1201, 1301, 1401, 1501. Said first connecting component may be connected to a connecting aspect of a protective apparatuses 102, 302, 402, 602 as shown herein.

Without limitation, said first connecting component may have an inner diameter of between approximately one inch and approximately five inches, preferably three inches. However, those of skill in the art will appreciate that alternative configurations of mechanical collars enabled by this disclosure may include first connecting components having inner diameters that are less than about one inch or greater than about five inches after having the benefit of this disclosure and without limitation. Said inner diameter may be of a size sufficient to accommodate a protective apparatus such as a protective apparatus or other accessory or attachment that has a corresponding outer diameter so as to allow for such protective apparatus or other accessory or attachment being received by the first connecting component.

As noted, mechanical collars enabled by the present disclosure may comprise a second connecting component 401, 702, 802, 902, 1102, 1202, 1302, 1402, 1600.

Mechanical collars enabled by the present disclosure may be connected, at the second connecting component, to an additional component or accessory, for example and without limitation, an articulating mechanical arm. According to such an embodiment, said mechanical arm may be used to facilitate positioning of the mechanical collar and the protective apparatus or other device with which the mechanical collar may be engaged. For example, where the second connecting component is connected to an articulating mechanical arm, said mechanical arm may be utilized to position a protective apparatus relative to a patient seated in a dentist's or other healthcare provider's office over whom said protective apparatus has been placed.

As reflected in FIGS. 4, 7-9, 12, and 16, the second connecting component may comprise a series of two substantially circular passages. The second substantially circular passage may be located within the first substantially circular passage.

In one illustrative embodiment, the second connecting component may comprise a first substantially circular passage 1203 having an inner diameter of between approximately one inch and approximately six inches, without limitation.

The second connecting component may comprise a second substantially circular passage 1204 that may have an outer diameter less than the inner diameter of the first substantially circular passage 1203. Exemplary arrangements of the two substantially circular passages relative one another and relative to the mechanical collar generally are depicted in FIGS. 4, 7-9, 12, and 16.

In one illustrative embodiment, the first substantially circular passage may have an inner diameter of between approximately one inch and approximately six inches, while the second substantially circular passage may have an outer diameter less than the inner diameter of the first substantially circular passage. However, those of skill in the art will appreciate that alternative configurations of mechanical collars enabled by this disclosure may include second connecting components having a first substantially circular passage with an inner diameter that is less than about one inch or greater than about six inches, and wherein the second substantially circular passage has an outer diameter less than the inner diameter of first substantially circular passage, after having the benefit of this disclosure and without limitation.

The second connecting component may further comprise a C-clamp 1303, 1403, 1502 located adjacent to, or otherwise near, the first substantially circular passage and the second substantially circular passage.

Mechanical collars enabled by the present disclosure may comprise a covering 1701, 1801 sufficient to hide from view cables, brake hoses, and/or similar items that may be used in connection with operation of a protective apparatuses when such a protective apparatus is engaged with a mechanical collar of the present disclosure. Illustrative, non-limiting, embodiments of mechanical collars of the present disclosure that include such coverings are depicted in FIGS. 17-18 submitted herewith.

Mechanical collars as contemplated herein may allow for rotating a protective apparatus when the collar is engaged with such protective apparatus. In one illustrative embodiment, mechanical collars of the present disclosure may allow for such rotatability by engaging with a rotating member located within a connecting aspect of a protective apparatus. Such rotatability may be effectuated through such mechanical means as would be readily appreciated by those of skill in the art. Such mechanical means may include, by way of example, bushing, including, for example, acetal homopolymer bushing. When engaged with a protective apparatus, mechanical collars of the present disclosure may allow for rotation of said protective apparatus at least 170° in both directions.

While the present disclosure refers to a first substantially circular passage and a second substantially circular passage, those of skill in the art will appreciate that said passages may, alternatively, define an elliptical or oval interior space.

While various aspects have been described in the above disclosure, the description of this disclosure is intended to illustrate and not limit the scope of the invention. The invention is defined by the scope of the claims and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the claims.

1. A mechanical collar comprising:
a first connecting component; and
a second connecting component;
wherein the first connecting component and the second connecting component are adjoined to one another;
and wherein the second connecting component comprises a first substantially circular passage and a second substantially circular passage;
wherein the second substantially circular passage has an outer diameter less than the inner diameter of the first substantially circular passage.

2. The mechanical collar of claim 1, wherein the mechanical collar is comprised of a material selected from the group consisting of the following: steel, metal, polycarbonate, polyphenylsulfone, and polyethylene.

3. The mechanical collar of claim 2, wherein the mechanical collar is comprised of steel.

4. The mechanical collar of claim 1, wherein the first connecting component has a diameter of between approximately one inch and approximately five inches.

5. The mechanical collar of claim 1, wherein the first substantially circular passage has an inner diameter of between approximately one inch and approximately six inches, and wherein the second substantially circular passage has an outer diameter less than the inner diameter of the first substantially circular passage.

6. The mechanical collar of claim 1, wherein the second connecting component further comprises a C-clamp.

7. The mechanical collar of claim 1, further comprising a covering locatable adjacent to the second connecting component.

8. A system for mitigating the risk of transmission of an infectious disease comprising:
a protective apparatus;
a mechanical collar component, wherein the mechanical collar component comprises:
a first connecting component; and
a second connecting component;
wherein the first connecting component and the second connecting component are adjoined to one another;
and wherein the second connecting component comprises a first substantially circular passage and a second substantially circular passage;
wherein the second substantially circular passage has an outer diameter less than the inner diameter of the first substantially circular passage;
wherein the protective apparatus is operatively attached to the first connecting component of the mechanical collar component; and
a mechanical arm component operatively attached to the second connecting component of the mechanical collar component.

9. The system of claim 8, wherein the mechanical collar component is comprised of a material selected from the group consisting of the following: steel, metal, polycarbonate, polyphenylsulfone, and polyethylene.

10. The system of claim 9, wherein the mechanical collar component is comprised of steel.

11. The system of claim 8, wherein the first connecting component has a diameter of between approximately one inch and approximately five inches.

12. The system of claim 8, wherein the first substantially circular passage has an inner diameter of between approximately one inch and approximately six inches, and wherein the second substantially circular passage has an outer diameter less than the inner diameter of the first substantially circular passage.

13. The system of claim 8, wherein the second connecting component further comprises a C-clamp.

14. The system of claim 8, wherein the mechanical collar component further comprises a covering locatable adjacent to the second connecting component.

15. A method for mitigating the risk of transmission of an infectious disease comprising:
providing a protective apparatus;
providing a mechanical collar component, wherein the mechanical collar component comprises:
a first connecting component; and
a second connecting component;
wherein the first connecting component and the second connecting component are adjoined to one another;
and wherein the second connecting component comprises a first substantially circular passage and a second substantially circular passage;
wherein the second substantially circular passage has an outer diameter less than the inner diameter of the first substantially circular passage;
wherein the protective apparatus is operatively attached to the first connecting component of the mechanical collar component; and
providing a mechanical arm component, wherein such mechanical arm component is operatively attached to the second connecting component of the mechanical collar component.

16. The method of claim 15, wherein the mechanical collar component is comprised of a material selected from the group consisting of the following: steel, metal, polycarbonate, polyphenylsulfone, and polyethylene.

17. The method of claim 15, wherein the first connecting component has a diameter of between approximately one inch and approximately five inches.

18. The method of claim 15, wherein the first substantially circular passage has an inner diameter of between approximately one inch and approximately six inches, and wherein the second substantially circular passage has an outer diameter less than the inner diameter of the first substantially circular passage.

19. The method of claim 15, wherein the second connecting component further comprises a C-clamp.

20. The method of claim 15, wherein the mechanical collar component further comprises a covering locatable adjacent to the second connecting component.
